(19) United States
(12) Patent Application Publication
(10) Pub. No.: US 2016/0015328 A1
(43) Pub. Date: Jan. 21, 2016

(54) PHYSICAL PROPERTIES CONVERTER

(71) Applicant: Sony Corporation, Tokyo (JP)

(72) Inventors: Staffan Dahlberg, Oxie (SE); Fredrik Permo, Vackelsang (SE)

(21) Appl. No.: 14/335,183

(22) Filed: Jul. 18, 2014

(51) Int. Cl. A61B 5/00; A61B 5/11; A61B 5/0205; A61B 5/145

(52) U.S. Cl. CPC A61B 5/744; A61B 5/14546; A61B 5/14542; A61B 5/1113; A61B 5/02055; A61B 5/486; A61B 5/6801; A61B 5/0002; A61B 5/4872; A61B 5/6898; A61B 5/72; A61B 5/024

(57) ABSTRACT

Aspects described herein relate to translation of physiological properties of a user's body to digital properties of a virtual object. The proposed technology also relates to a method and device for handling the translation. According to one aspect, the disclosure relates to a method, performed in an electronic device including communication circuitry and processing circuitry for translating the properties. The method includes the steps of receiving information representing physiological properties of the user's body, the information being collected using at least one sensor, calculating digital properties associated with a virtual object, wherein the virtual object is a virtual representation of the user, based on the physiological properties of the user's body and providing the digital properties to the virtual object in a virtual environment. Aspects described herein further relate to an electronic device for implementing the method and a corresponding computer program.

### TECHNICAL FIELD

The proposed technology relates to translation of physiological properties of a user's body to digital properties of a virtual object. The proposed technology also relates to a method and device for handling the translation.

### BACKGROUND

Today there is an arising health problem concerning too little physical activity. It has been showed that for many people the problem is closely linked with digital gaming. Too much sedentary behavior in front of a screen is not good for the health and this problem can be seen from low ages. Many young individuals do not see the actual or direct gain in having a fit/healthy body in today's society. The negative effects of the increased screen time behavior is often first seen in older ages and it might be hard to change to an active/healthy lifestyle because of future health effects. Trends are showing that this public health risk is not likely to decrease in the near future.

Because the negative effects of this behavior are often first seen in older ages it is hard to motivate people to change to a more active/healthier lifestyle.

There is a need to motivate people with a lot of screen time to a more active and healthy life style.

### SUMMARY

An aspect of the present disclosure is to provide a method and an electronic device which seek to mitigate, alleviate, or eliminate the above-identified deficiency in the art and to provide a solution where the people are motivated to be more active and healthy because of direct benefits in the virtual world. The present disclosure proposes a method and device for visualizing physiological properties of a user's body.

More specifically, the invention proposes a method and device for translation of physiological properties of a user's body to digital properties of a virtual object. To increase people's motivation to live a healthier and more active lifestyle their physiological attributes are converted to digital properties of objects in games and other software. In other words, our physical training in real life will effect and give benefits in the digital world. Users get an instant gratification, an additional purpose of working out and a motivator to gain a healthier lifestyle.

The present disclosure is defined by the appended claims. Various advantageous embodiments of the disclosure are set forth by the appended claims as well as by the following description and the accompanying drawings.

According to some aspects of the disclosure, it provides for a method of visualizing physiological properties of a user's body, wherein the method is configured to be performed in an electronic device, the electronic device comprising communication circuitry and processing circuitry.

According to some aspects the method comprises the steps of:
receiving information representing physiological properties of the user's body, the information being collected using at least one sensor;
calculating digital properties associated with a virtual object, wherein the virtual object is a virtual representation of the user, based on the physiological properties of the user's body; and
providing the digital properties to the virtual object in a virtual environment.

According to some aspects of the disclosure, it provides for an electronic device, for visualizing physiological properties of a user's body, comprising:
communication circuitry for wireless communication;
processing circuitry configured to:
   receive, using the communication circuitry or from at least one sensor of the electronic device, information representing physiological properties of the user's body, the information being collected using at least one sensor;
   calculate digital properties associated with a virtual object, wherein the virtual object is a virtual representation of the user, based on the physiological properties of the user's body; and
   provide the digital properties to the virtual object in a virtual environment.

In other words a method and a device for transferring a user's training and physique to a virtual object is provided and he/she receives instant benefits in the virtual world from his/her training. Thus, users are motivated to exercise so that they receive instant gratification. The physiological properties of the user's body are used to calculate digital properties of the virtual object and the digital properties dictate the behavior of the virtual object.

According to some aspects the digital properties are provided to the virtual object as physical attributes of the virtual object. Hence, the physiological properties of the user's body are translated to physical attributes of the virtual object. If the user performs a lot of agility training the virtual object can for example jump higher.

According to some aspects the virtual object is an object or a structure in a computer program. In other words, digital properties are provided to an object or structure in a computer program.

According to some aspects the virtual object in the virtual environment is a character in an interactive virtual environment. When the virtual object is a character in an interactive virtual environment it is easy for the user to see the results of his/her training in the virtual world, for example in a game.

According to some aspects the digital properties comprise points associated with the physiological properties of the user's body and wherein providing the digital properties to the virtual object in the virtual environment further comprises that the user assigns the points to at least two different physical attributes of the virtual object. An advantage with a point system is that a user which is performing cardio training and a user which is performing weight lifting can receive the same amounts of points. The users can then choose to assign the points to any attributes of the virtual object. For example, if the virtual object is a character in a shoot 'em up game the user may want to assign the character speed to run fast and enough strength to hold heavy weaponry. With the point system, users who are exercising very differently can participate in the same games under the same conditions.

According to some aspects the at least one sensor comprises at least one of: a pulse sensor, a galvanic skin response sensor, a blood sugar sensor, a blood lipids sensor, a body temperature sensor, an oxygen level sensor, a fluid level sensor, an electrolyte sensor, a motion sensor, a location sensor and/or a body fat sensor. In other words, the at least one sensor is a sensor that measures any kind of physiological property of the user.

According to some aspects the processing circuitry is further configured to perform the method comprising the step of storing the calculated digital properties in a memory of the electronic device. The digital properties may thereafter be used multiple times and be provided to several virtual objects. Storing the digital properties makes it possible for the user to choose to provide old digital properties to a virtual object. In this way a parent can for example play a game with his/her child and use his/her digital properties from when he/she was the same age as his/her child.

According to some aspects the processing circuitry is further configured to perform the method comprising the step of requesting physiological properties of the user's body from a second electronic device comprising the at least one sensor. The physiological properties of the user's body are the physiological properties of a user of the second electronic device. Thus, the electronic device is not required to be the device that collects the physiological properties.

According to some aspects the second electronic device is a wearable device. Since a wearable device is by definition a device worn on a user's body, it is a well suited device to use for collecting the physiological properties.

According to some aspects the processing circuitry is further configured to perform the method comprising the step of requesting physiological properties of the user's body from a database. When requesting the physiological properties from a database there are multiple options on how to collect them. There may for example be several devices comprising sensors that have collected the properties and then sent them to the database.

According to some aspects the physiological properties of a user's body are the physiological properties of a user of a third electronic device comprising the at least one sensor, wherein the third electronic device configured to transmit the physiological properties of the user's body to the database. The third electronic device is for example a wearable device, a pedometer or a smartphone or anything that may be used to collect the physiological properties of the user.

According to some aspects the at least one sensor is located in the electronic device. Thus, it is the electronic device that both collects physiological properties and performs the method.

According to some aspects providing the digital properties to the virtual object in the virtual environment further comprises sending the digital properties to a fourth electronic device. In other words, an external electronic device may use the digital properties on virtual objects. For example a gaming consol may receive the properties and use them on virtual objects in games.

According to some aspects providing the digital properties to the virtual object in the virtual environment further comprises updating an object, structure and/or variable in a computer program defined by the processing circuitry. Thus, the digital properties provided to the virtual object are used directly in the electronic device.

According to some aspects of the disclosure, it provides for a computer program, comprising computer readable code which, when run on an electronic device, causes the electronic device to perform the method according to above.

With the above description in mind, aspects of the present disclosure motivate people with a lot of screen time to a more active and healthy life style as previously described.

### BRIEF DESCRIPTION OF THE DRAWINGS

The present invention will be more readily understood through the study of the following detailed description of the embodiments/aspects together with the accompanying drawings, of which:

FIGS. 1, 2, 3, 4A, 4B, 5 and 6 illustrate examples of electronic devices.

FIG. 7 is a flow chart illustrating the proposed method, performed in the electronic device.

FIGS. 8A, 8B and 9 illustrate a user of an electronic device performing physical activities.

FIGS. 10, 11, 12, 13, 14, 15, 16, 17, 18, 19 and 20 illustrate examples of communicating electronic devices.

### DETAILED DESCRIPTION

Aspects of the present disclosure will be described more fully hereinafter with reference to the accompanying drawings. The method and device disclosed herein can, however, be realized in many different forms and should not be construed as being limited to the aspects set forth herein. Like numbers in the drawings refer to like elements throughout.

The terminology used herein is for the purpose of describing particular aspects of the disclosure only, and is not intended to limit the disclosure. As used herein, the singular forms “a”, “an” and “the” are intended to include the plural forms as well, unless the context clearly indicates otherwise.

The term wearable and wearable device are used interchangeably and are referring to a wireless device which is worn somewhere on the body of a user. Examples of wearables are watches, wristbands, headsets, headbands, necklaces, necklace-headsets, ankle bands etc.

Unless otherwise defined, all terms (including technical and scientific terms) used herein have the same meaning as commonly understood by one of ordinary skill in the art to which this disclosure belongs. It will be further understood that terms used herein should be interpreted as having a meaning that is consistent with their meaning in the context of this specification and the relevant art and will not be interpreted in an idealized or overly formal sense unless expressly so defined herein.

As used herein, the term “electronic device” includes portable communication devices, portable radio communication devices, servers, gaming consols and wearables. A portable communication device or a portable radio communication device may be referred to below as a smartphone and includes all electronic equipment, including, but not limited to, capable of using voice and/or data communication. As will be appreciated, the disclosure may be used with mobile phones, other phones, smartphones, personal digital assistants (PDAs), computers, other communication devices, laptops, tablets, wearables, servers, gaming consols, etc.

The present disclosure proposes a method of visualizing physiological properties of a user's body, wherein the method is configured to be performed in an electronic device 1, the electronic device 1 comprising communication circuitry 2 and processing circuitry 3. Furthermore, en electronic device is provided which performs the method. The physiological properties are the measured functions and activities of the user. Mechanical, physical, and biochemical processes may be monitored using at least one sensor and recorded as physiological properties. The physiological properties are for example how far the user has been running how fast, the blood pressure of the user and/or the stress level of the user. More examples of physiological properties and examples of how they are collected are described below.

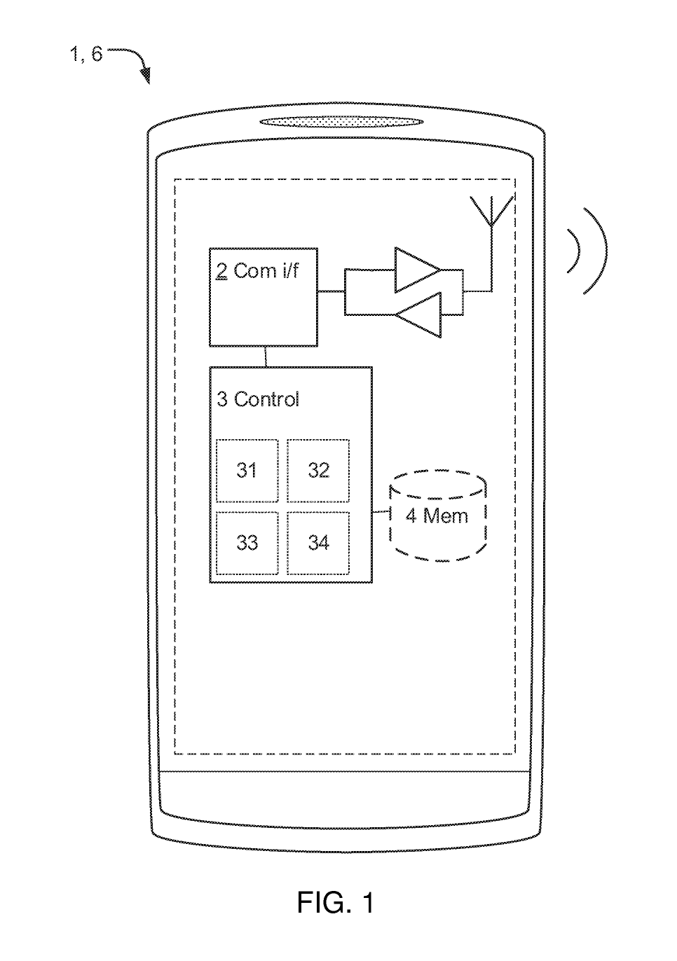

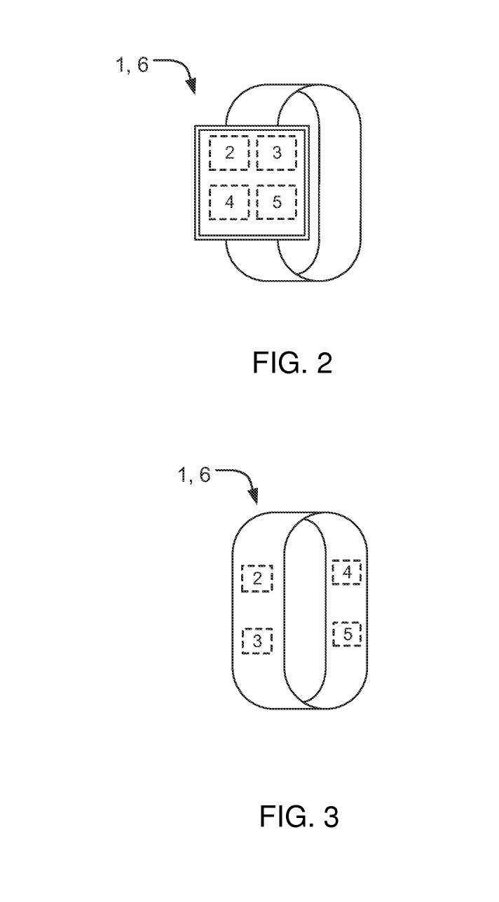

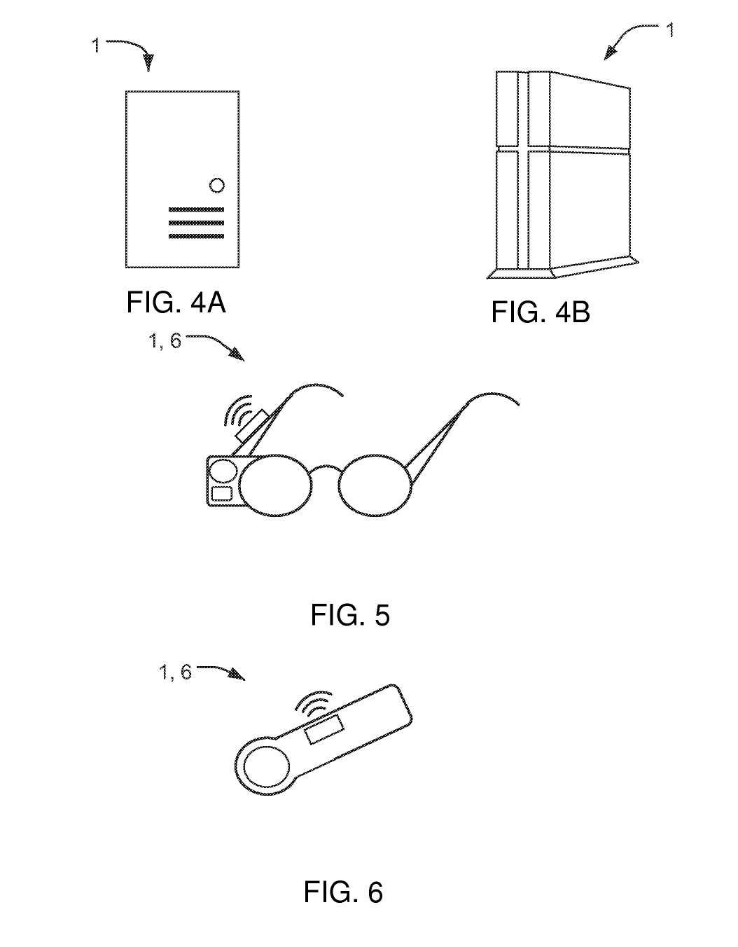

FIGS. 1, 2, 3, 4A, 4B, 5 and 6 illustrates examples of electronic devices 1. In FIG. 1 the wireless device is illustrated as a mobile communication device, such as a smartphone. In FIG. 2 the electronic device is illustrated as a wearable device, such as a watch. In FIG. 3 the electronic device is a wristband. FIGS. 4a and 4b illustrates the electronic device as a server and a gaming consol respectively. In FIG. 5 the electronic device is a pair of wearable glasses and in FIG. 6 the electronic device is a headset.

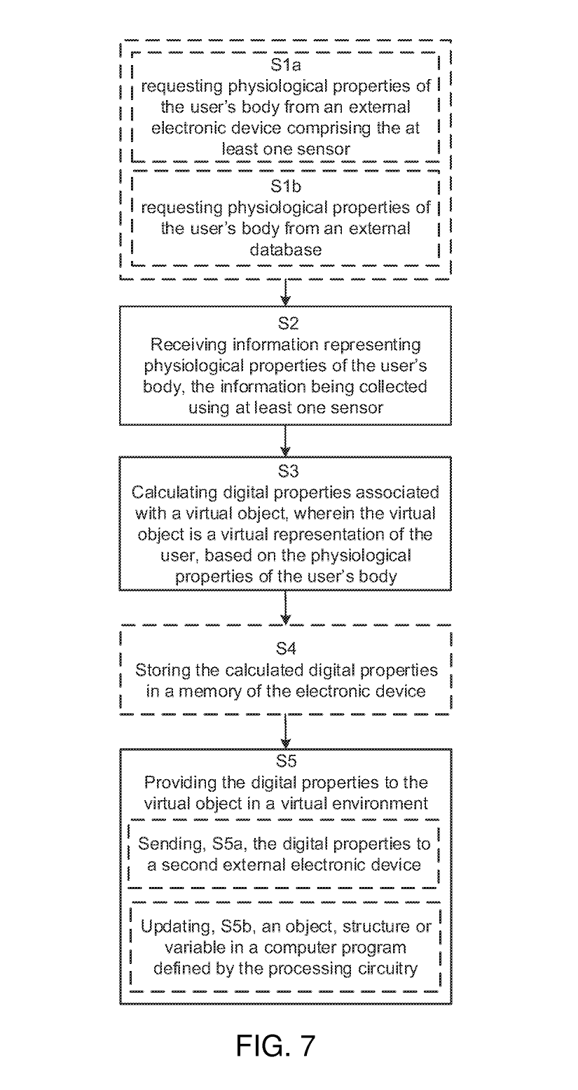

FIG. 7 is a flow diagram depicting example operations which may be taken by the electronic devices of FIGS. 1 to 6.

It should be appreciated that FIG. 7 comprise some operations which are illustrated with a solid border and some operations which are illustrated with a dashed border. The operations which are comprised in a solid border are operations which are comprised in the broadest example embodiment. The operations which are comprised in a dashed line are example embodiments which may be comprised in, or a part of, or are further operations which may be taken in addition to the operations of the broader example embodiments. It should be appreciated that the operations need not be performed in the order shown in the figure. Furthermore, it should be appreciated that not all of the operations need to be performed.

The proposed technique will now be briefly described referring to FIGS. 1, 2, 3, 4A, 4B, 5, 6 and 7. As previously discussed, the disclosure provides for a method and device for transferring physiological properties of a user's body to a virtual object.

The method comprises the first step of receiving S2 information representing physiological properties of the user's body, the information being collected using at least one sensor 5. The processing circuitry 3 is configured to receive the information using the communication circuitry or from at least one sensor of the electronic device. In other words, the information is either received from an external device comprising at least one sensor or directly from at least one sensor of the electronic device. According to some aspects the processing circuit comprises a receiver 31 for receiving the information. The user is a user of an electronic device which comprises the at least one sensor which collects physiological properties. Note that the method which is being described here does not need to be performed on the same electronic device as the electronic device which collects the physiological properties of the user. Examples of this will be given below.

The method further comprises the second step of calculating S3 digital properties associated with a virtual object, wherein the virtual object is a virtual representation of the user, based on the physiological properties of the user's body. The processing circuitry 3 is configured to calculate the digital properties. According to some aspects the processing circuitry comprises a calculator 32 for calculating. Thus, digital properties are derived from the physiological properties of the user's body. The digital properties are calculated so that they are compatible with a virtual object. A virtual object is an object in any kind of virtual environment. That is, a virtual object has a form which has a constant or variable shape.

The method further comprises the third step of providing S5 the digital properties to the virtual object in a virtual environment. The processing circuitry 3 is configured to providing the digital properties to the virtual object. According to some aspects the processing circuitry comprises a provider 33 for providing the digital properties. The virtual environment is a computer-generated, two or three-dimensional representation of a setting in which users of the technology perceives themselves to be and within which interaction takes place; also called virtual landscape, virtual space, virtual world. The physiological properties of the user's body are used to calculate digital properties of the virtual object and the digital properties dictate the behavior of the virtual object. In other words, the digital properties provided to the virtual object dictates how the virtual object moves and functions in the virtual environment. Providing digital properties to a virtual object will be done differently depending on the virtual object. It will be up to the developer of the virtual object and the virtual environment to decide how to apply digital properties based on the physiological properties of a user's body to the virtual object. An example of how to convert physiological propereties to digital properties is as follows. Strenght may be measured in different groups of muscles, such as biceps, triceps, shoulders, forearms, chest, back, abs, quads, hamstrings, calves. Every kilo that a user can lift during an exercise with seven repetitions will give one exercise point P. So if a user can do seven repetitions with 15 kg in an arm curl biceps exercise he/she gets 15P. A user may for example exercise so that the total strength score is as follows: biceps 15P, triceps 13P, shoulders 10P, forearms 5P, chest 62P, back 50P, abs 40P, quads 60P, hamstrings 40P and calves 23P which gives a total strength of 318P. Another example of measuring strength is that the total muscle strain is measured and a higher strain gives a higher exercise point. Sleep may be measured in amount of minutes of sleep. An optimal amount of sleep is considered to be 8 hours, that is 480 minutes. 480 minutes may give 480P and every minute less gives 1P less. It might be that the optimal sleep time changes. It is also possible that every minute above 480P gives −1P to prevent users from oversleeping or if the user has had one short sleeping period he/she may compensate for that and sleep longer the next time. Sleeping time may be measured using for example motion sensors. Sleeping time may also be inserted into the electronic device by the user. Note that the physiological properties of a user's body does not need to be visible to other users since the information may be regarded as personal and sensitive.

The calculation of the digital properties is automatic and not something that users sees.

With the above described steps a way to transfer a user's training and physique to a virtual object is provided and he/she receives instant benefits in the virtual world from his/hers training. For example, if a user is a fast runner and goes out running with an electronic device comprising at least one sensor which tracks the running, he/she will get a fast virtual object in the virtual world. The virtual object may also be more stable since it will not get winded if moving fast. Thus, users are motivated to exercise so that they receive instant gratification. When the user is for example playing a game in which he/she is controlling the virtual object provided the digital properties, he/she will see improvements in the movements of the virtual object when he/she has exercised. If a user is playing a first person shooter game the character the user is playing will for example be faster if the user is fast and aim faster if the user is more fit. In a first person shooter game the users plays a character in first person view and the object is usually to shoot competing players with different weapons. If a user is strong the character will for example be able to carry more and heavier weapons and have more health. If a user is well character the user will for example get bigger radar, faster firing rate, faster recharge, faster weapon change, faster recovery after running, etc. Characters in games will of course need to have a basic set of properties that exercise of the user affects. The exercise of a user that gives physiological properties will be added on top of those basic properties. Note that the user utilizing the virtual object in a virtual environment does not need to be the user from which the physiological properties have been collected. A person may use the physiological properties of a known soccer star, provided that the soccer star has made the data available, so that other users may play virtual soccer against a virtual object in the form of a virtual soccer player with the physiological properties of that star. Another example of using the method is when a group of people use their own physiological properties and form a virtual soccer team. The team may them play against other teams using their own physiological properties. This will of course work in any game with any kind of team play.

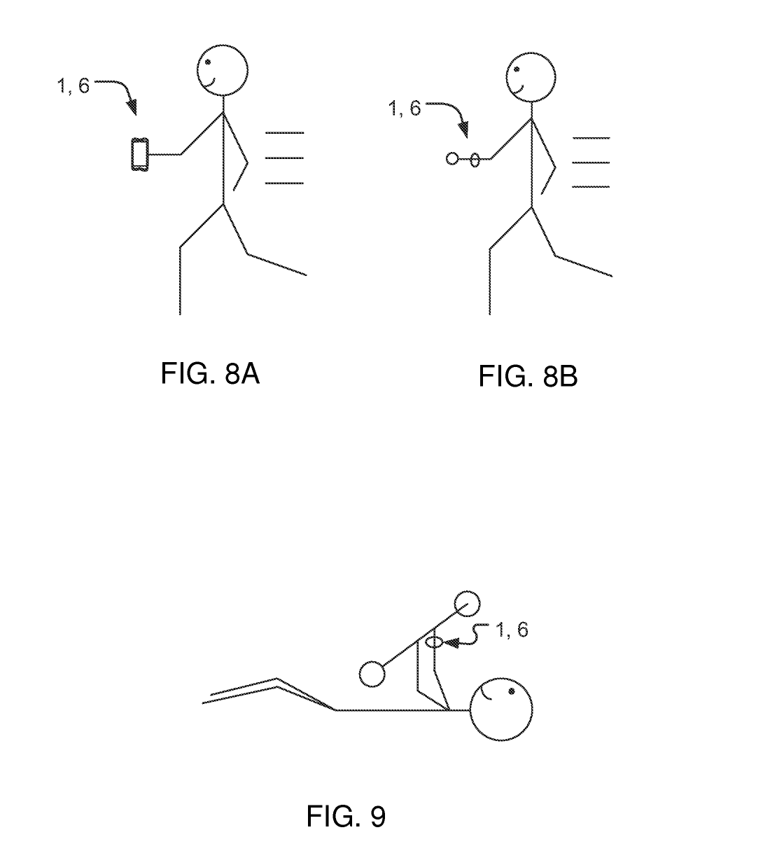

FIGS. 8A, 8B and 9 illustrates an example when a user with an electronic device is collecting sensor data. In FIG. 8A a user collects running data with a smartphone. The smartphone uses for example a location sensor such as GPS and a motion sensor to count the number of steps. In FIG. 8B the user is wearing a wearable device which is collecting the physiological properties. The smartphone and wearable in the figures are used to collect the physiological properties. According to some aspects the smartphone or the wearable is the electronic device 1 performing the method according to above. According to some aspects the smartphone or wearable device is a second electronic device 6 which sends collected physiological properties to the electronic device 1. In FIG. 9 the user is wearing a wearable device and performs weight lifting. An example of how to collect physiological properties of a user's body is that a user uses an electronic device with a Near Field Communication (an NFC) reader to scan NFC tags located on different weights and training machines in a gym to collect data regarding for example the weight of the weights or the settings of a machine. Then the electronic device uses motion sensors such as an accelerometer and/or a gyroscope to count the number of repetitions and the speed of which the user lifts the weight or uses the machine. The information about the weights and/or machines together with the information from the motion sensors comprises the physiological properties of the user's body.

According to some aspects the digital properties are provided to the virtual object as physical attributes of the virtual object. Hence, the physiological properties of the user's body are translated to physical attributes of the virtual object. Physical attributes of the virtual object are attributes which decides how the virtual object moves and behaves in the virtual environment. In other words, physical attributes of the virtual object defines for example the speed, strength, agility, height, weight, etc. of the virtual object in the virtual environment. Thus, if the user performs a lot of agility training the virtual object can for example jump higher in the virtual environment.

According to some aspects the virtual object is an object or a structure in a computer program. In other words, digital properties are provided to an object or structure in a computer program. The object or structure comprises a collection of variables and/or functions defining the virtual object. The computer program is the program for generating and running the virtual environment.

According to some aspects the virtual object in the virtual environment is a character in an interactive virtual environment. A interactive virtual environment is, as described above, a computer-generated, two or three-dimensional representation of a setting in which users of the technology perceives themselves to be and within which interaction takes place; in another word interacts. The character is a character with which the user interacts in the virtual environment. How a user interacts in an interactive virtual environment is well known to a person skilled in the art and will not be described further. When the virtual object is a character in an interactive virtual environment it is easy for the user to see the results of his/her training in the virtual world, for example in a game. A character in a game which becomes more fit when you are will serve as a motivator for users to exercise. The basic idea is that if you for example get stronger in real life, your virtual character will also get stronger, if you do a lot of cardio vascular training your digital character will gain endurance and if you not had enough sleep, this will also affect your character, but in a negative way.

According to some aspects the digital properties comprise points associated with the physiological properties of the user's body and wherein providing S5 the digital properties to the virtual object in the virtual environment further comprises that the user assigns the points to at least two different physical attributes of the virtual object. In virtual environments such as games it is common for the virtual objects that the users control for interaction has a number of “stats”, or statistics that specify specific attributes, advantages and/or disadvantages, powers, skills and/or traits of the character. The user earns points when exercising and assigns these points to physical attributes of the virtual object. The physical attributes are for example strength, agility, eye sight, hearing, stealth, charisma, intelligence etc. According to some aspects the points may also be assigned to give the virtual object skills and/or powers or similar. The user which assigns the point is the user from which the physiological properties have been collected. According to some aspects the user which assigns the points is a user different from the user from which the physiological properties have been collected. An example of such a case is that users may for example use the physiological properties of known athletes. An advantage with a point system is that a user which is performing cardio training and a user which is performing weight lifting can receive the same amounts of points. The users can then choose to assign the points to any attributes of the virtual object. For example, if the virtual object is a character in a shoot 'em up game the user may want to assign the character speed to run fast and enough strength to hold heavy weaponry. With the point system, users who are exercising very differently can participate in the same games under the same conditions.

Note that a combination of a point system and an automatic system may be used. Some physiological properties, for example amount of running exercise, may be directly translated to speed of the virtual object, while other properties, for example number if times a specific weight has been lifted, is transferred into point.

According to some aspects exercise is transferred into the game as currency of the virtual object. This may also be used in combination with the method described in the disclosure. An example of this is that a user equipped with an electronic device which counts the number of steps taken by the user receives those steps as currency of a virtual object.

According to some aspects the at least one sensor comprises at least one of: a pulse sensor, a galvanic skin response sensor, a blood sugar sensor, a blood lipids sensor, a body temperature sensor, an oxygen level sensor, a fluid level sensor, an electrolyte sensor, a motion sensor, a location sensor and/or a body fat sensor. In other words, the at least one sensor is a sensor that measures any kind of physiological property of the user. A pulse sensor is for example an Electrocardiography, ECG, device which picks up electrical impulses generated by the polarization and depolarization of cardiac tissue and translates into a waveform. The waveform is then used to measure the rate and regularity of heartbeats. A galvanic skin response, GSR, sensor measures the electrical conductance of the skin, which varies depending on the amount of sweat-induced moisture on the skin. Sweat is controlled by the sympathetic nervous system, so skin conductance is used as an indication of psychological or physiological stress. A blood sugar sensor, blood lipids sensor, oxygen level sensor, fluid level sensor, body fat sensor and electrolyte sensor are for example sensors positioned under the skin which measures and transmits the result to a receiver. The body temperature sensor is for example a thermometer. The motion sensor is for example an accelerometer and/or gyroscope. The location sensor is for example a Global Positioning System, GPS.

The physiological properties of the user's body are thus collected using, for example, one of a combination of the above mentioned sensors. Another example of how to collect physiological properties of a user's body is if a user has been certified to enter a specific competition which required a certain physique, then the required physique may be used as part of the user's physiological properties.

Note that parameters measured by several sensors may be combined to give one physiological property. For example, amount of cardio training, blood pressure and how rested the user is may affect the alertness of the virtual object. One sensor may also give more than one physiological property. For example blood pressure may affect rest and alertness.

According to some aspects the processing circuitry 3 is further configured to perform the method comprising the step of storing S4 the calculated digital properties in a memory 4 of the electronic device. The digital properties may thereafter be used multiple times and be provided to several virtual objects. Storing the digital properties makes it possible for the user to choose to provide old digital properties to a virtual object. In this way a parent can for example play a game with his/her child and use his/her digital properties from when he/she was the same age as his/her child. It would also provide the possibility to see which user was the best of all times in virtual environments. For example if Mike Tyson and Muhammad Ali had logged their physiological properties when they were at their best physical level, they could use their properties in a boxing game to see which of them was the best. In other words, you may use the stats from your “glory days” and compete with other people when they also were in their best shapes. A lot of people that have been training for a long time know that it takes a lot of hard work to stay fit over time and you need consistent maintenance. To give some credit to people that have been in really good shape, but for some reasons, injuries or for other reasons haven't been able to work out for a while, they can still live on old merits.

According to some aspects the processing circuitry 3 is further configured to perform the method comprising the step of requesting S1a physiological properties of the user's body from a second electronic device 6 comprising the at least one sensor. The physiological properties of the user's body are the physiological properties of a user of the second electronic device 6. Thus, as also discussed earlier, the electronic device is not required to be the device that collects the physiological properties. For example, the electronic device is a server or a gaming device and the second electronic device is a wearable device or a smartphone. For example, the electronic device is a smartphone and the second electronic device is a wearable device.

According to some aspects the second electronic device 6 is a wearable device. Since a wearable device is by definition a device worn on a user's body, it is a well suited device to use for collecting the physiological properties using at least one sensor. When the second electronic device is a wearable device, the electronic device is a smartphone, a server or a gaming consol.

According to some aspects the processing circuitry 3 is further configured to perform the method comprising the step of requesting S1b physiological properties of the user's body from a database 9. When requesting the physiological properties from a database there are multiple options on how to collect them. There may for example be several devices comprising sensors that have collected the physiological properties and then sent them to the database. For example, a user may have a wearable that collects physiological properties using a thermometer and a smartphone that collects physiological properties using a motion sensor.

According to some aspects the physiological properties of a user's body are the physiological properties of a user of a third electronic device 7 comprising at least one sensor, wherein the third electronic device is configured to transmit the physiological properties of the user's body to the database. The third electronic device is for example a wearable device, a pedometer or a smartphone or anything that may be used to collect the physiological properties of the user. Note that the second and the third electronic devices may be the same device. According to some aspects the electronic device receives physiological properties from multiple sources, such as from a database and from a second electronic device. The physiological properties received from multiple sources either represents different physical attributes of the user's body or, if the properties represent the same thing, the newest data is used when calculating the digital properties.

According to some aspects the at least one sensor is located in the electronic device 1. Thus, it is the electronic device that both collects physiological properties and performs the method. According to one aspect the physiological properties are collected from multiple sources such as from a second electronic device, from a database and/or from sensors in the electronic device. As previously discussed the second electronic device and the database will also collect the physiological data with at least one sensor.

According to some aspects providing S5 the digital properties to the virtual object in the virtual environment further comprises sending S5a the digital properties to a fourth electronic device 8. In other words, an external electronic device may use the digital properties on virtual objects. For example a gaming consol may receive the properties and use them on virtual objects in games. According to some aspects the second, third and/or the fourth electronic device are the same device but they may also be different devices.

According to some aspects providing S5 the digital properties to the virtual object in the virtual environment further comprises updating S5b an object, structure and/or variable in a computer program defined by the processing circuitry. Thus, the digital properties provided to the virtual object are used directly in the electronic device. This is the case when the electronic device is for example a smartphone or a gaming consol which runs the virtual environment

The method according to above provides digital properties, representing a user's physiological properties, to virtual objects in virtual environments. According to some aspects both existing and new games may use the digital properties on virtual objects as a plug in so that users may play games using their own physiological properties in any existing or new game, both on computer games and on consol games. According to some aspects the disclosure works as a plugin to a lot of different games and/or other existing software. So the conversion of physiological properties of a user's body to digital properties of a virtual object does not have to be locked to a specific game or software.

According to some aspects, the disclosure is a base for complete new types of games that are more closely connecting real life with user's virtual life. For instance, users may get a mission in a game that you need to pick up a virtual token in Malmö City center and you are only allowed to get there by foot and as quick as possible.

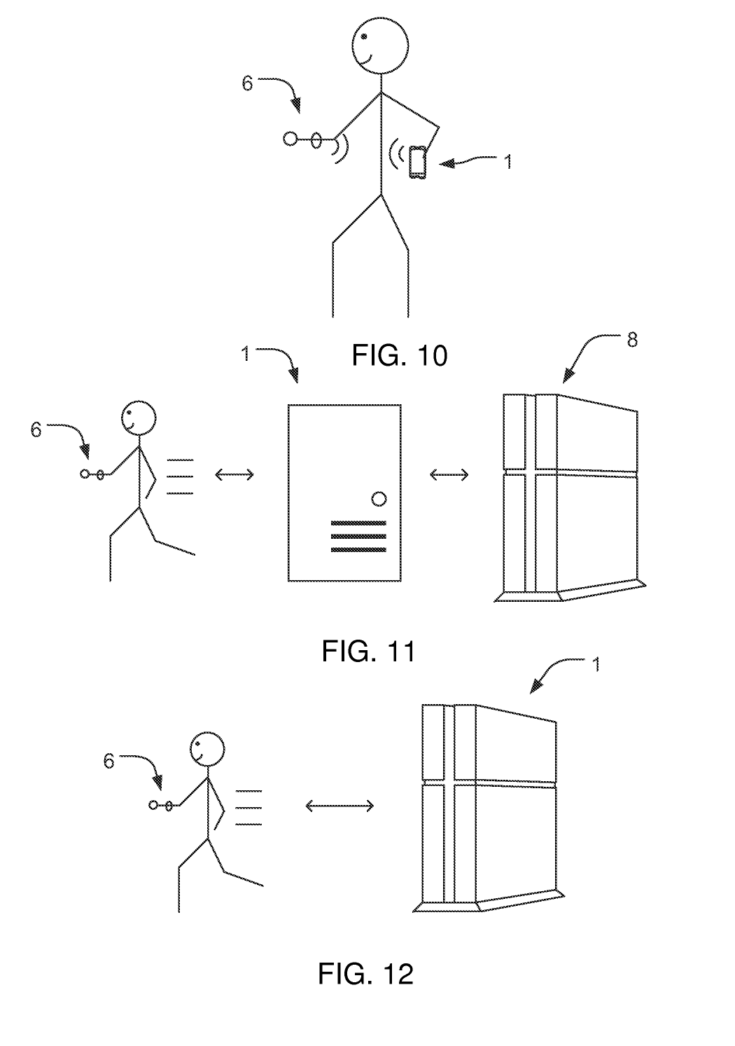

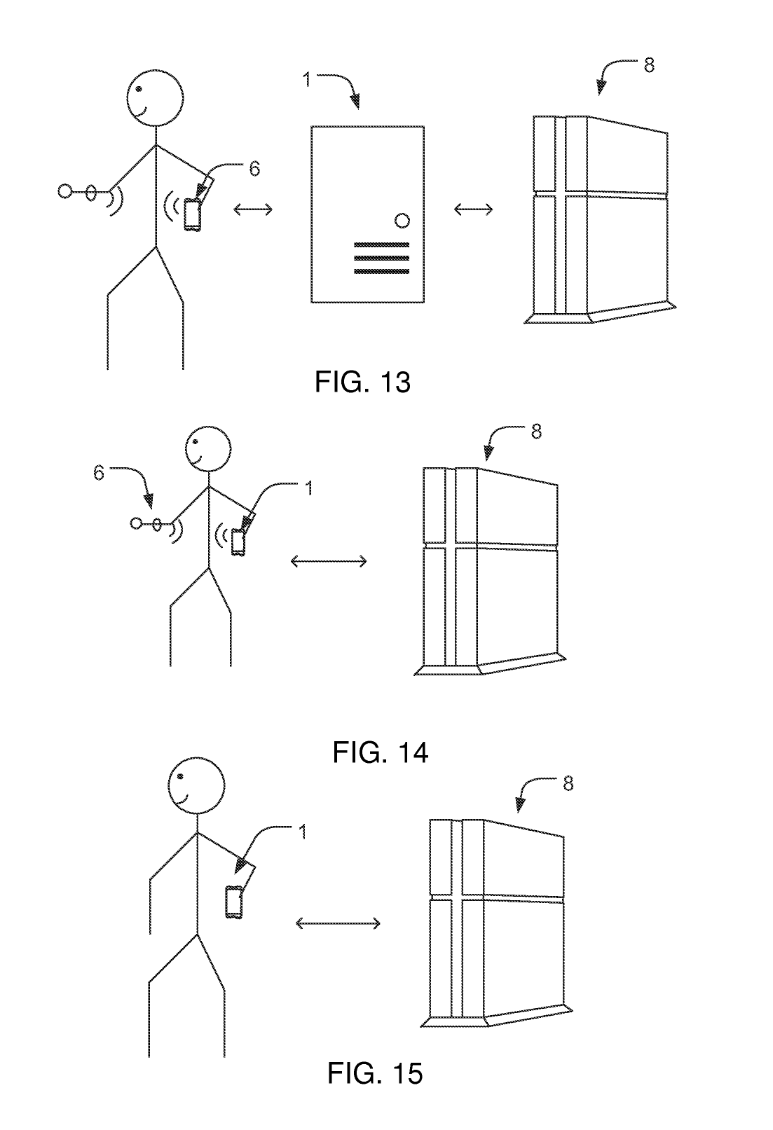

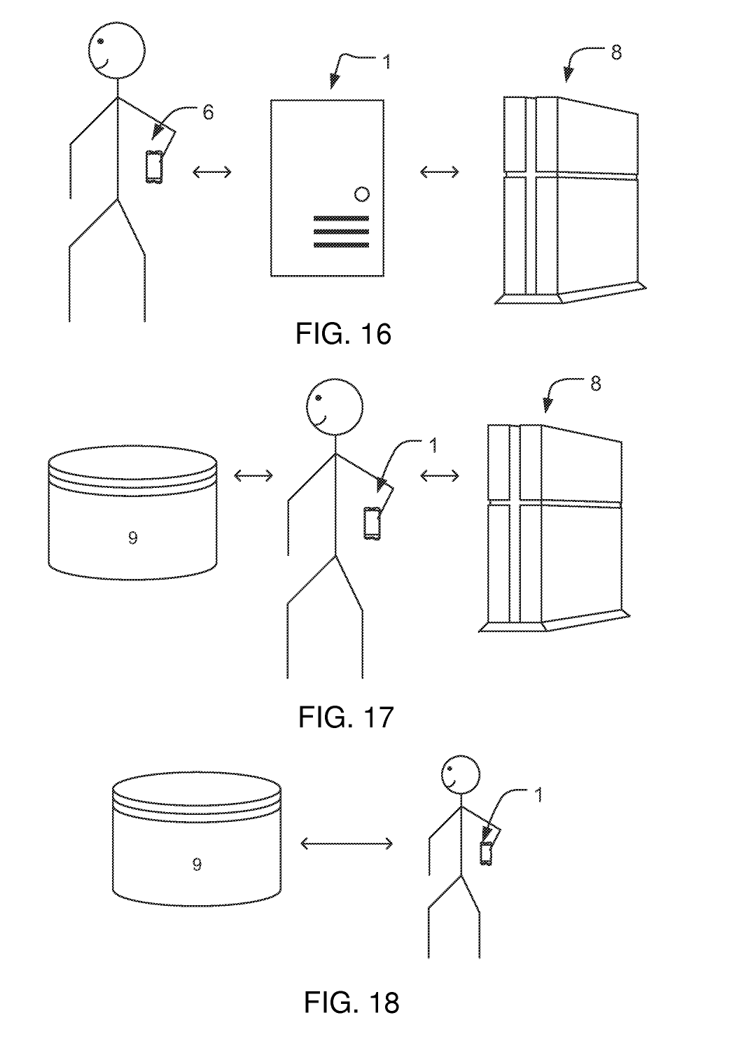

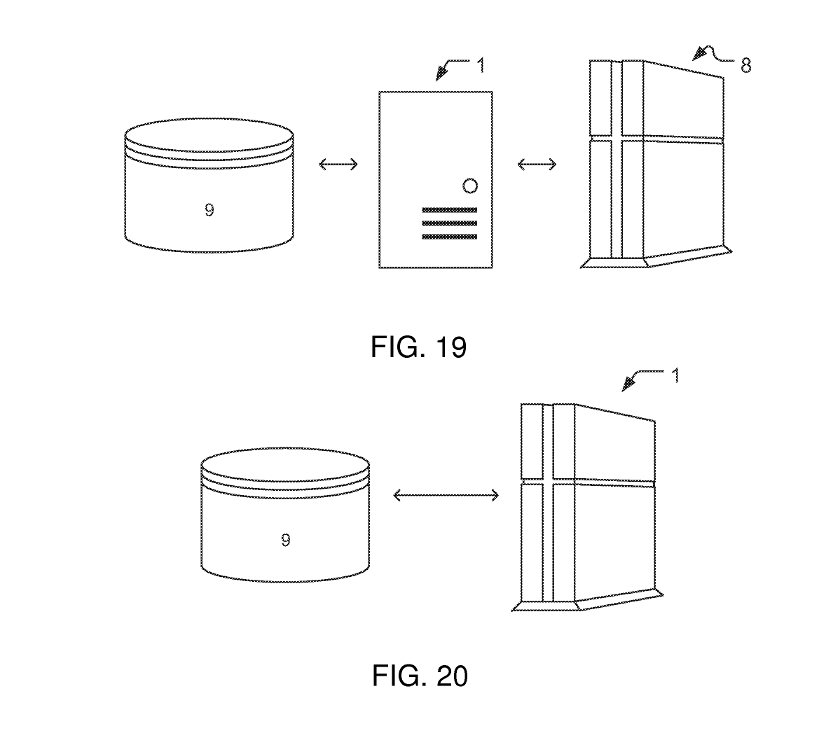

FIGS. 10 to 20 illustrate examples of which electronic devices are involved depending on the set up of the devices. In FIG. 10 a smartphone is the electronic device 1 and a wearable is the second electronic device 6. In FIG. 11 a server is the electronic device 1, a wearable is the second electronic device 6 and a gaming consol is the fourth electronic device 8. In FIG. 12 a gaming consol is the electronic device 1 and a wearable device is the second electronic device 6. In FIG. 13 a server is the electronic device 1, a smartphone is the second electronic device 6, a gaming consol is the fourth electronic device 8 and a wearable device may be connected to the smartphone to collect physiological properties. In FIG. 14 a smartphone is the electronic device 1, a wearable device is the second electronic device 6 and a gaming consol is the fourth electronic device 8. In FIG. 15 a smartphone is the electronic device 1 and a gaming consol is the fourth electronic device 8. In FIG. 16 a server is the electronic device 1, a smartphone is the second electronic device 6 and a gaming consol is the fourth electronic device 8. In FIG. 17 a smartphone is the electronic device 1, a database 9 provides the physiological properties and a gaming consol is the fourth electronic device 8. In FIG. 18 a smartphone is the electronic device 1 and a database 9 provides the physiological properties. In FIG. 19 a server is the electronic device 1, a database 9 provides the physiological properties and a gaming consol is the fourth electronic device 8. In FIG. 20 a gaming consol is the electronic device 1 and a database 9 provides the physiological properties.

In some implementations and according to some aspects of the disclosure, the functions or steps noted in the blocks can occur out of the order noted in the operational illustrations. For example, two blocks shown in succession can in fact be executed substantially concurrently or the blocks can sometimes be executed in the reverse order, depending upon the functionality/acts involved. Also, the functions or steps noted in the blocks can according to some aspects of the disclosure be executed continuously in a loop.

The description of the example embodiments provided herein have been presented for purposes of illustration. The description is not intended to be exhaustive or to limit example embodiments to the precise form disclosed, and modifications and variations are possible in light of the above teachings or may be acquired from practice of various alternatives to the provided embodiments. The examples discussed herein were chosen and described in order to explain the principles and the nature of various example embodiments and its practical application to enable one skilled in the art to utilize the example embodiments in various manners and with various modifications as are suited to the particular use contemplated. The features of the embodiments described herein may be combined in all possible combinations of methods, apparatus, modules, systems, and computer program products. It should be appreciated that the example embodiments presented herein may be practiced in any combination with each other.

It should be noted that the word “comprising” does not necessarily exclude the presence of other elements or steps than those listed and the words “a” or “an” preceding an element do not exclude the presence of a plurality of such elements. It should further be noted that any reference signs do not limit the scope of the claims, that the example embodiments may be implemented at least in part by means of both hardware and software, and that several “means”, “units” or “devices” may be represented by the same item of hardware.

The various example embodiments described herein are described in the general context of method steps or processes, which may be implemented according to some aspects by a computer program, comprising computer readable code which, when run on an electronic device, causes the electronic device to perform the method according to above. The computer program, embodied in a computer-readable medium, includes computer-executable instructions, such as program code, executed by computers in networked environments. A computer-readable medium may include removable and non-removable storage devices 4 including, but not limited to, Read Only Memory, ROM, Random Access Memory, RAM, compact discs, CDs, digital versatile discs, DVD, etc. Generally, program modules may include routines, programs, objects, components, data structures, etc. that performs particular tasks or implement particular abstract data types. Computer-executable instructions, associated data structures, and program modules represent examples of program code for executing steps of the methods disclosed herein. The particular sequence of such executable instructions or associated data structures represents examples of corresponding acts for implementing the functions described in such steps or processes.

In the drawings and specification, there have been disclosed exemplary embodiments. However, many variations and modifications can be made to these embodiments. Accordingly, although specific terms are employed, they are used in a generic and descriptive sense only and not for purposes of limitation, the scope of the embodiments being defined by the following claims.

1. A method of visualizing physiological properties of a user's body, wherein the method is configured to be performed in an electronic device, the electronic device comprising communication circuitry and processing circuitry, the method comprising:
receiving information representing physiological properties of the user's body, the information being collected using at least one sensor;
calculating digital properties associated with a virtual object, wherein the virtual object is a virtual representation of the user, based on the physiological properties of the user's body; and
providing the digital properties to the virtual object in a virtual environment.

2. The method according to claim 1, wherein the digital properties are provided to the virtual object as physical attributes of the virtual object.

3. The method according to claim 1, wherein the virtual object is an object or a structure in a computer program.

4. The method according to claim 3, wherein the virtual object in the virtual environment is a character in an interactive virtual environment.

5. The method according to claim 1, wherein the digital properties comprise points associated with the physiological properties of the user's body and wherein providing the digital properties to the virtual object in the virtual environment further comprises that the user assigns the points to at least two different physical attributes of the virtual object.

6. The method according to claim 1, wherein the at least one sensor comprises at least one of: a pulse sensor, a galvanic skin response sensor, a blood sugar sensor, a blood lipids sensor, a body temperature sensor, an oxygen level sensor, a fluid level sensor, an electrolyte sensor, a motion sensor, a location sensor and/or a body fat sensor.

7. The method according to claim 1, further comprising:
storing the calculated digital properties in a memory of the electronic device.

8. The method according to claim 1, further comprising:
requesting physiological properties of the user's body from a second electronic device comprising the at least one sensor,
wherein the physiological properties of the user's body are the physiological properties of a user of the second electronic device.

9. The method according to claim 8, wherein the second electronic device is a wearable device.

10. The method according to claim 1, further comprising:
requesting physiological properties of the user's body from a database.

11. The method according to claim 10, wherein the physiological properties of a user's body are the physiological properties of a user of a third electronic device comprising the at least one sensor, wherein the third electronic device is configured to transmit the physiological properties of the user's body to the database.

12. The method according to claim 1, wherein the at least one sensor is located in the electronic device.

13. The method according to claim 1, wherein providing the digital properties to the virtual object in the virtual environment further comprises sending the digital properties to a fourth electronic device.

14. The method according to claim 1, wherein providing the digital properties to the virtual object in the virtual environment further comprises updating an object, structure and/or variable in a computer program defined by the processing circuitry.

15. An electronic device, for visualizing physiological properties of a user's body, comprising:
communication circuitry for wireless communication;
processing circuitry configured to:
receive, using the communication circuitry or from at least one sensor of the electronic device, information representing physiological properties of the user's body, the information being collected using at least one sensor;
calculate digital properties associated with a virtual object, wherein the virtual object is a virtual representation of the user, based on the physiological properties of the user's body; and
provide the digital properties to the virtual object in a virtual environment.

16. The electronic device according to claim 15, wherein the digital properties are provided to the virtual object as physical attributes of the virtual object.

17. The electronic device according to claim 15, wherein the virtual object is an object or a structure in a computer program.

18. The electronic device according to claim 17, wherein the virtual object in the virtual environment is a character in an interactive virtual environment.

19. The electronic device according to claim 15, wherein the digital properties comprise points associated with the physiological properties of the user's body and wherein to provide the digital properties to the virtual object in the virtual environment further comprises that the user assigns the points to at least two different physical attributes of the virtual object.

20. The electronic device according to claim 15, wherein the at least one sensor comprises at least one of: a pulse sensor, a galvanic skin response sensor, a blood sugar sensor, a blood lipids sensor, a body temperature sensor, an oxygen level sensor, a fluid level sensor, an electrolyte sensor, a motion sensor, a location sensor and/or a body fat sensor.

21. The electronic device according to claim 15, wherein the processing circuitry is further configured to:
store the calculated digital properties in a memory of the electronic device.

22. The electronic device according to claim 15, wherein the processing circuitry is further configured to:
request physiological properties of the user's body from a second electronic device comprising the at least one sensor,
wherein the physiological properties of the user's body are the physiological properties of a user of the second electronic device.

23. The electronic device according to claim 22, wherein the second electronic device is a wearable device.

24. The electronic device according to claim 15, wherein the processing circuitry is further configured to:
request physiological properties of the user's body from a database.

25. The method according to claim 25, wherein the physiological properties of a user's body are the physiological properties of a user of a third electronic device comprising at least one sensor, wherein the third electronic device is configured to transmit the physiological properties of the user's body to the database.

26. The electronic device according to claim 15, wherein the at least one sensor is located in the electronic device.

27. The electronic device according to claim 15, wherein to provide the digital properties to the virtual object in the virtual environment further comprises to send the digital properties to a fourth electronic device.

28. The electronic device according to claim 15, wherein to provide the digital properties to the virtual object in the virtual environment further comprises to update an object, structure and/or variable in a computer program defined by the processing circuitry.

29. A non-transitory computer readable medium storing a computer program comprising computer readable code which, when run on an electronic device, causes the electronic device to perform the method as claimed in claim 1.
